(19) United States
(12) Patent Application Publication
(10) Pub. No.: US 2020/0129223 A1
(43) Pub. Date: Apr. 30, 2020

(54) ELECTROSURGICAL DEVICE CONSOLE

(71) Applicant: AERIN MEDICAL, INC., Sunnyvale, CA (US)

(72) Inventors: Michael Angeles, Alberta (CA); Andrew Frazier, Sunnyvale, CA (US); Fred Dinger, Austin, TX (US)

(21) Appl. No.: 16/668,678

(22) Filed: Oct. 30, 2019

Related U.S. Application Data
(60) Provisional application No. 62/753,469, filed on Oct. 31, 2018.

(51) Int. Cl. A61B 18/12

(52) U.S. Cl. CPC A61B 18/1206; A61B 2018/00327

(57) ABSTRACT

A console for an electrosurgical device may include a housing, an energy generator in the housing, a computer processor in the housing, a connector on the housing, for connecting the console with an energy delivery treatment device, a touchscreen display on the housing, and a non-transitory computer readable medium in the computer processor. The computer readable medium may contain computer-executable programming instructions for providing an active display on the touchscreen display. The active display may include a central circle, a countdown timer in the central circle for counting down a total time remaining in an energy delivery procedure, and an outer ring around the central circle, configured to indicate the total time remaining in the energy delivery procedure, a remaining time in an energy delivery stage of the energy delivery procedure, and a remaining time in a cooling stage of the energy delivery procedure.

### CROSS-REFERENCE TO RELATED APPLICATIONS

The present application claims priority to U.S. Provisional Patent Application Ser. No. 62/753,469, titled “Electrosurgical Device Console,” filed Oct. 31, 2019, the full disclosure of which is hereby incorporated by reference.

### FIELD

The present application is related to surgical devices and methods. More specifically, the application is related to an electrosurgery system console and user interface.

### BACKGROUND

Surgical devices that use energy to treat tissue in the body are used for a wide variety of procedures, to treat many different conditions and injuries. The assignee of the present application, for example, has developed a number of devices, systems and methods for treating airway tissue, specifically nasal upper airway tissue in some embodiments, by delivering energy to the tissue. For example, in some embodiments, an energy delivery device is inserted into a patient's nostril to deliver energy (and sometimes mechanical force) to tissue in the nose. This type of energy delivery treatment, for example radiofrequency (RF) energy delivery, may be used to reshape cartilage and/or other structures in the nose and/or to change other properties of tissues such as nasal mucosa. The treatments may address any of a wide variety of conditions, just two examples of which are nasal valve insufficiency (which may cause difficulty breathing through the nose) and chronic rhinitis (runny nose).

Electrosurgical devices typically include some type of stylus, wand, applicator or other handheld component for applying the energy to the patient (i.e., for performing the surgical procedure) and a generator, which may sometimes be referred to as a “box” or “console.” The generator is attached to the applicator via a cord, to provide energy to the applicator. In some cases, the generator may also send and/or receive signals to and from the applicator (for example, treatment algorithms, tissue temperature measurements, etc.). Although many different electrosurgical consoles have been developed over the years, it would still be advantageous to have an improved console. For example, an improved console might be specifically designed for a particular type of energy delivery device or a particular set of procedures.

### BRIEF SUMMARY

This application describes various aspects and embodiments of an energy delivery system console (or “box”). More particularly, this application describes an improved user interface for an electrosurgical console/generator, which allows a physician user to monitor and alter treatment parameters for a particular treatment stylus, applicator, catheter or the like. The console is primarily described herein as a radiofrequency (RF) electrosurgical generator with a user interface/display. In alternative embodiments, however, the console, or a modified version thereof, may be part of a system that delivers other types of energy, such as but not limited to heat, laser, microwave, cryogenic cooling, DC current or ultrasound.

In one aspect of the present disclosure, a console for an electrosurgical device may include: a housing; an energy generator in the housing; a computer processor in the housing; a connector on the housing, for connecting the console with an energy delivery treatment device; a touchscreen display on the housing; and a non-transitory computer readable medium in the computer processor, containing computer-executable programming instructions for providing an active display on the touchscreen display. The active display includes a central circle, a countdown timer in the central circle for counting down a total time remaining in an energy delivery procedure, and an outer ring around the central circle. The outer ring indicates the total time remaining in the energy delivery procedure, a remaining time in an energy delivery stage of the energy delivery procedure, and a remaining time in a cooling stage of the energy delivery procedure.

In one embodiment, the energy generator is a radiofrequency (RF) generator, configured to deliver monopolar and/or bipolar RF energy to the energy delivery treatment device. In some embodiments, the outer ring changes colors in a clockwise direction to indicate elapsed time of the energy delivery procedure. Optionally, the active display may further include a start/stop button and a custom treatment button, both of which are configured to be activated by touch. Optionally, the active display may further include a temperature display, an energy on indicator, and/or a procedure count indicator, indicating a number of procedures completed using the energy delivery treatment device.

In some embodiments, the active display may further include a custom treatment button that, when activated, causes the display screen to display a custom treatment screen. The custom treatment screen may include, for example, a set cooling time window, a set power window, a set temperature window and/or a set procedure time window.

In some embodiments, the connector on the housing is configured to accept only one predefined type of energy delivery treatment device. Optionally, the non-transitory computer readable medium in the computer processor may include an energy delivery treatment device identifier for identifying the energy delivery treatment device that is connected to the console.

In another aspect of the present disclosure, an active display on a touchscreen of an electrosurgical device may include a central circle, a countdown timer in the central circle for counting down a total time remaining in an energy delivery procedure, and an outer ring around the central circle. The outer ring indicates the total time remaining in the energy delivery procedure, a remaining time in an energy delivery stage of the energy delivery procedure, and a remaining time in a cooling stage of the energy delivery procedure. The outer ring may change colors in a clockwise direction to indicate elapsed time of the energy delivery procedure and/or may include a line that moves around the outer ring in a clockwise direction to count down the total time remaining in the energy delivery procedure. All optional features described above may be included in the active display, according to various embodiments.

In another aspect of the present disclosure, a method for performing an energy delivery therapy on nasal airway tissue in a patient may involve: activating a console attached to an energy delivery stylus; advancing a distal end of the energy delivery stylus into a nostril of the patient; delivering energy from the energy delivery stylus to treat the nasal airway tissue; and watching a display on a display screen of the console. The display on the console may include a central circle, a countdown timer in the central circle for counting down a total time remaining in the energy delivery therapy, and a moving outer ring around the central circle, configured to indicate the total time remaining in the energy delivery therapy, a remaining time in an energy delivery stage of the energy delivery therapy, and a remaining time in a cooling stage of the energy delivery therapy. Finally, the method involves removing the energy delivery stylus from the nostril after completion of the cooling stage.

In some embodiments, delivering energy involves delivering radiofrequency energy, and the energy delivery stylus is a radiofrequency stylus. In some embodiments, delivering energy involves starting the delivery of energy by touching a start/stop button on the display screen and/or depressing a foot pedal coupled with the console.

The method may also involve observing a displayed temperature, a displayed number of procedures performed with the energy delivery stylus, an energy on indicator, and/or a stylus identifier on the display screen. The method may optionally involve touching a custom treatment button on the display screen to switch to a custom treatment display and setting a temperature, a power, a therapy time, and/or a cooling time, by touching at least one control button on the custom treatment display. Ion some embodiments, the method may include viewing an error display on the display screen and touching a back button on the display screen to return to a previous screen. The method may also optionally include viewing a settings display on the display screen and adjusting brightness and/or volume of the console by touching at least one controller on the display screen.

Optionally, the method may include, before removing the energy delivery stylus from the nostril: moving the distal end of the energy delivery stylus to a different location in the nasal airway; delivering energy from the energy delivery stylus to treat nasal airway tissue at the different location; and watching the display on the display screen of the console. The method may further include repeating the moving, delivering and watching steps as often as desired to perform the energy delivery therapy on the nasal airway tissue. In some embodiments, the method may include attaching the energy delivery stylus to the console and waiting for the console to confirm that the energy delivery stylus is approved for use, where the console activates after it confirms the energy delivery stylus is approved.

These and other aspects and embodiments of the disclosure are described in further detail below, in relation to the attached drawing figures.

### BRIEF DESCRIPTION OF THE DRAWINGS

FIG. 1 is a front perspective view of an RF electrosurgical system for treating airway tissue, according to one embodiment;

FIG. 2 is a rear perspective view of the console of the electrosurgical system of FIG. 1;

FIGS. 3A-3C are top, front and side views, respectively, of the console shown in FIGS. 1 and 2;

FIG. 4 is a screenshot of a standby screen display that may be displayed on an electrosurgical console, according to one embodiment;

FIG. 5 is a screenshot of a default main screen display on an electrosurgical console, as it appears before the electrosurgery treatment has started, according to one embodiment;

FIG. 6 is a screenshot of a default main screen display on an electrosurgical console, as it appears during an RF delivery stage of an electrosurgery treatment, according to one embodiment;

FIG. 7 is a screenshot of a default main screen display on an electrosurgical console, as it appears during a cooling stage of an electrosurgery treatment, according to one embodiment;

FIG. 8 is a screenshot of a custom treatment settings screen display on an electrosurgical console, as it appears during a cooling stage of an electrosurgery treatment, according to one embodiment;

FIG. 9 is a screenshot of a fault screen display on an electrosurgical console, according to one embodiment;

FIG. 10 is a screenshot of an error screen display on an electrosurgical console, according to one embodiment;

FIG. 11 is a screenshot of a settings screen display on an electrosurgical console, according to one embodiment; and

FIG. 12 is a flow diagram of a method of performing an electrosurgical treatment, using a console as described in the present application, according to one embodiment.

### DETAILED DESCRIPTION

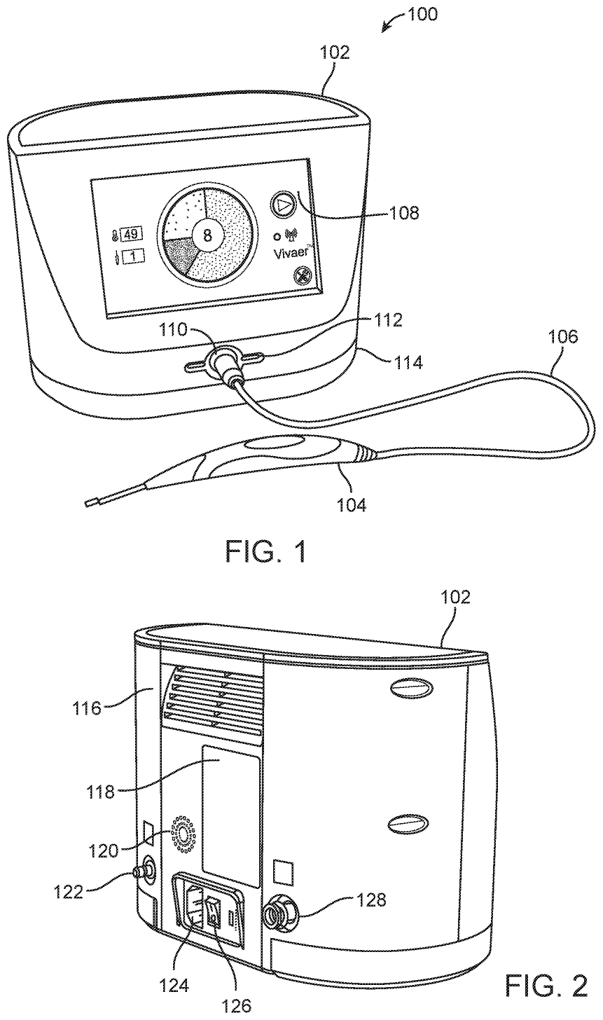

Referring to FIG. 1, one example of an RF electrosurgical system 100 is illustrated. As mentioned above, in alternative embodiments the electrosurgical system 100 may be modified to provide and deliver any other suitable type of energy, in addition to or instead of RF. In this example, the RF electrosurgical system 100 includes a console 102, an RF delivery stylus 104, and a cable 106 connecting the stylus 104 to the console 102. The console 102 houses an RF generator, a processor, and various electronics, none of which is visible in FIG. 1. The word “console,” in this disclosure, is meant to encompass the terms “generator,” “box,” “controller,” and any other commonly used terms to describe an electrosurgical system console or generator. The parts of the console 102 that are visible in FIG. 1 include a touchscreen display 108, a stylus connection port 110, an “RF ON” indicator light 112, and a bottom ring 114. The touchscreen display 108 serves as the main user interface for interacting with the console 102 and will be described further below. The stylus connection port 110 allows a connection end (or “connector”) of the stylus 104 to be plugged into it. The stylus connection port 110 can be configured to accept only the connection end of the stylus 104 and can be configured to not accept or work with counterfeit or other devices. In some embodiments, electronics inside the console 102 may include a stylus identification safety feature that identifies the stylus 104 when it is plugged into the stylus connection port 110. Such a safety feature may, for example, automatically shut down (or disable powering on) the console 102, if a user tries to plug in a device other than the stylus 104.

The RF ON indicator light 112 indicates when RF energy is being delivered through the stylus connection port 110 to the stylus 104. The bottom ring 114, in this embodiment, lights up when the console 102 is powered on. This lighted ring 114 is an optional feature. Both the RF ON indicator light 112 and the lighted bottom ring 114 may have any color or colors of light. In one embodiment, for example, the RF ON indicator light 112 is blue, and the bottom ring 114 lights up with a white light. This is merely one example, however, and any suitable lighting configuration and combination of colors may be used in alternative embodiments.

FIG. 2 shows the back of the console 102. In this embodiment, the console 102 includes an air vent 116, a product label area 118, a speaker 120, an equipotential ground connection port 122, a main power connection port 124, a main power switch 126 and foot switch connection port 128. The equipotential ground connection port 122 is provided at the back of the device.

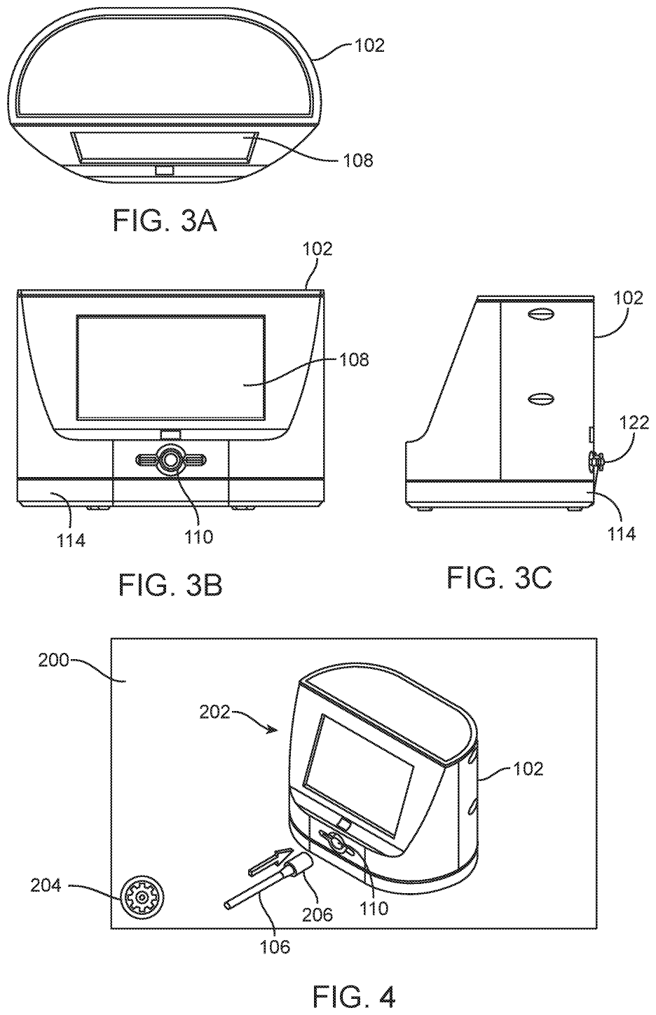

FIGS. 3A-3C are top, front and side views, respectively, of the console 102 shown in FIGS. 1 and 2.

Referring now to FIG. 4, a screenshot of one example of a standby image 200 that may be shown on the display 108 of the console 102 is illustrated. The standby image 200 may be displayed after the console 102 is powered on. In some embodiments, the console 102 may perform a power-on self-test before showing the standby image 200. The standby image includes a console image 202, a settings button 204 and an animated insert stylus image 206. The animated insert stylus image 206 shows the connector end of the stylus cable 106 moving toward the console stylus connection port 110. Thus, the image 200 graphically informs the user that the console 102 is ready for use, awaiting insertion of the connection end of the stylus cable 106 into the connection port 110.

As mentioned above, in some embodiments, the console 102 may be programmed to identify the stylus 104 inserted into its connection port 110 and accept only specified styluses 104, for security and patient safety purposes. For example, the console 102 may accept only one specific brand of stylus 104. The console 102 may also be capable of determining whether a stylus 104 is new or used and may only work or activate when a new stylus 104 is inserted. This feature may be very helpful in protecting patients, since used styluses 104 may provide substandard performance. In some embodiments, the console 102 may monitor the use of a particular inserted stylus 104, determine when the stylus 104 has completed a predetermined amount of usage, and deactivate the stylus 104 and/or the console 102 when the predetermined amount is achieved.

In some embodiments, the console 102 may permanently deactivate the stylus 104 when it has reached its maximum number of uses. For example, in a given procedure on a nasal airway of a patient, the stylus 104 might be designed to deliver 10-20 treatments of RF delivery, or more specifically in some embodiments 12-15 treatments. Each treatment may last, for example, 15-20 seconds, or more specifically 18 seconds, with 10-15 seconds of cool-down time. If a doctor administers a maximum number of treatments with a given stylus 104, such as 15 treatments in one example, the console 102 will turn off or go into a deactivated mode and may also permanently deactivate the stylus or permanently mark it as “used.” If a user tries to insert a used stylus 104 into the console 102, the console 102 will not turn on or activate, and it may also provide the user with a signal on the screen 108, such as “Error” or “Used Stylus” or “Unusable Stylus” or the like.

In some embodiments, as will be described further below, the console 102 may be designed for use with different types of styluses 104 for different procedures. For example, one embodiment of the console 102 may be used with one type of stylus 104 for a nasal valve remodeling procedure, a second type of stylus 104 for a chronic rhinitis treatment procedure, a third type of stylus 104 for a soft palate procedure and/or the like. When each specific type of stylus 104 is inserted, the console 102 is programmed to identify the stylus 104 and provide a treatment algorithm designed for that particular stylus type. Again, if a used or incompatible stylus 104 is inserted, the console 102 will not activate or provide energy to the stylus 104.

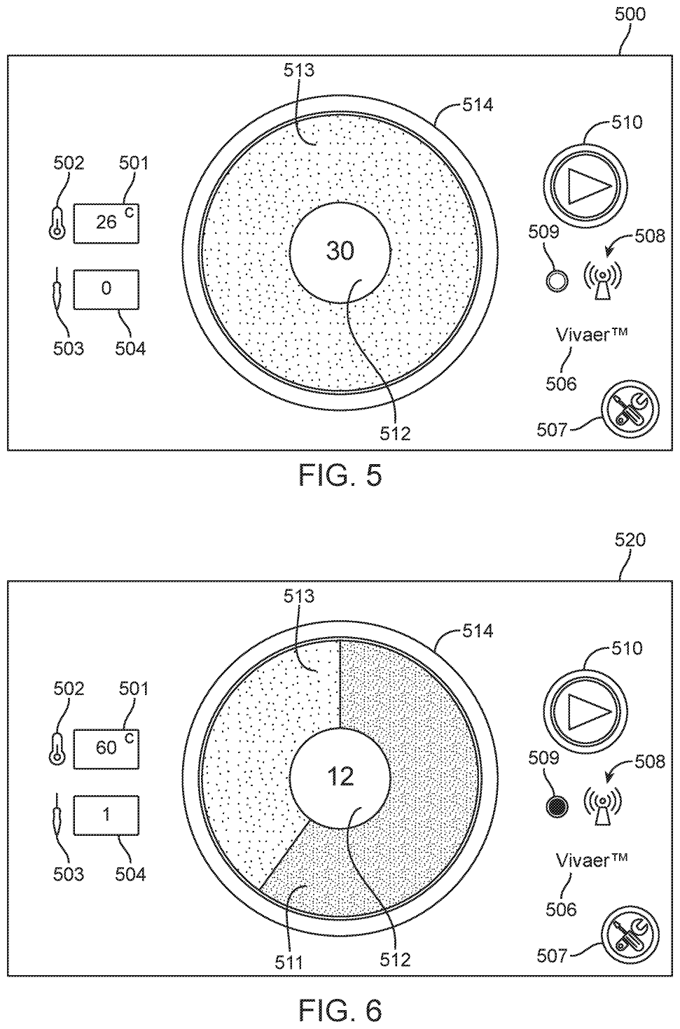

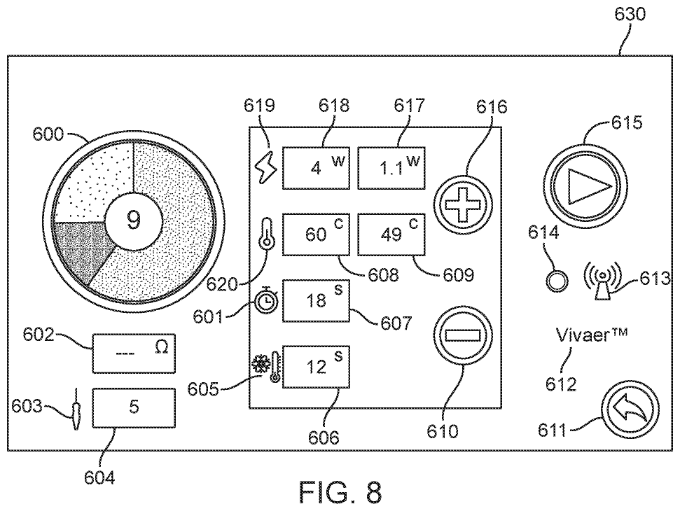

Referring now to FIG. 5, once the physician or other user has attached the stylus 104 to the console 102, the standby image of FIG. 4 may be replaced on the display 108 by a default main screen image 500. This main screen image 500 is what the user sees before the procedure has started. Under normal operating conditions, the user can select either default treatment settings or manual treatment settings. The default treatment settings are pre-loaded into the processor of the console 102 and do not require any additional settings inputs from the user. In some embodiments, it may be possible for the user to select from several sets of default settings. Operation under the default settings mode is described in relation to FIGS. 5-7, and operation under the custom settings mode is described in relation to FIG. 8.

FIG. 5 shows the default image 500 displayed on the display 108 of the console 102, once a valid stylus 104 is connected. In this embodiment, the default image includes a stylus temperature indicator 501, a temperature icon 502, a stylus icon 503, treatment number indicator 504, a stylus type indicator 506, a start/stop button 510, an RF ON indicator light 509, an RF icon 508, a custom treatment button 507 and a central, circular, graphical treatment progress display 514.

The graphical treatment progress display 514 has several portions, according to the embodiment shown in FIG. 5. First, there is a total treatment timer 512, which is displayed as a central circle with a counting down number, representing seconds (or alternatively minutes, or minutes and seconds) remaining in the current procedure. Here, for example, the treatment has not started yet, and the total treatment timer 512 shows a time of 30 seconds, thus indicating that at least the next tissue treatment will last 30 seconds total. Immediately surrounding the central circle total treatment timer 512 is a treatment time indication ring 513, which acts as an indicator of elapsed and remaining time in the procedure. In the default image 500 of FIG. 5, the treatment has not started, so the entire outer ring 513 is one initial color. The initial color of the ring 513 indicates time remaining in the treatment, which in this case takes up the entire ring 513.

Other indicators on the screenshot image 500 also show that the treatment has not yet started. For example, the RF ON indicator light 509 is not illuminated yet, because the console 102 is not yet delivering RF energy to the stylus 104. The treatment number indicator 504 shows that zero (“0”) treatments have been performed with the stylus 104 that is currently plugged into the console 102. And the temperature indicator 501 shows a stylus temperature of 26 degrees Celsius. To begin a treatment, the physician user will touch the start/stop button 510 on the touchscreen display 108.

In the embodiment of FIG. 5, the stylus 104 inserted into the console 102 is a VivAer® Nasal Airway Remodeling Stylus (www.aerinmedical.com), as indicated by the stylus type indicator 506. This embodiment of the console 102 may accept one or more other types of styluses 104, for example a stylus designed for treating chronic rhinitis and/or a stylus designed for treating soft palate tissue. If such an alternative type of stylus 104 is inserted, the console 102 will identify the stylus type and indicate the type via the stylus type indicator 506.

Referring now to FIG. 6, a later screenshot 520 of the default settings screen is illustrated. At this stage, RF energy is being delivered from the console 102 to the stylus 104, as indicated by the RF ON indicator light 509. The total treatment timer 512 of the graphical treatment progress display 514 shows that 12 seconds remain in the treatment—in other words, 18 seconds of the 30-second total time have elapsed. The outer ring 513 now includes a darker RF energy delivery time portion 511, designating the elapsed 18 seconds of RF energy delivery time. The lighter remaining portion of the ring 513 indicates the 12-second portion of the total treatment time that is still remaining. As the RF energy delivery stage of the treatment begins and progresses, the darker RF energy delivery time portion 511 sweeps clockwise around the outer ring 513, thus taking up more and more of the outer ring 513. In other words, the RF energy deliver indicator 511 starts at zero, at the twelve o'clock position on the ring 513, and moves around the ring in a clockwise direction. In this embodiment, treatment time (during which RF energy is delivered) is 18 seconds, and cool down time (during which RF energy is turned off) is 12 seconds. In alternative embodiments, any suitable alternative treatment times and cool down times may be used.

Other indicators that the treatment is in progress include the temperature indicator 501, showing a temperature of 60 degrees Celsius, and the treatment number indicator 504 showing that this is the first treatment being performed with the stylus 104 currently plugged into the console 102.

In some embodiments, the console 102 may be activated, and RF energy delivered to the stylus 104 in either of two ways—the start/stop button 510 may be touched, or a foot pedal coupled with the console 102 may be depressed. The RF ON indicator 509 lights up when the console 102 is delivering RF power. The stylus type connected indicator 506 indicates what type of stylus 104 is connected to the console 102, which in the example shown is a VivAer® Stylus. This indicator 506 may be useful in embodiments where the console 102 is configured for use with multiple different types of styluses. The stylus temperature indicator 501 shows the actual temperature of the distal, treatment end of the stylus 104. The treatment number indicator 504 displays the number of the treatment currently being completed with the stylus 104 that is attached to the console 102. Finally, the custom treatment button 507 allows the user to customize one or more treatment parameters. Touching the custom treatment button 507 will lead the user to a new display screen with different options. In alternative embodiments, the various icons and/or indicators on the default display 520 may be changed or moved. In some embodiments, one or more of the icons and/or indicators may be eliminated and/or additional indicators and/or buttons may be added.

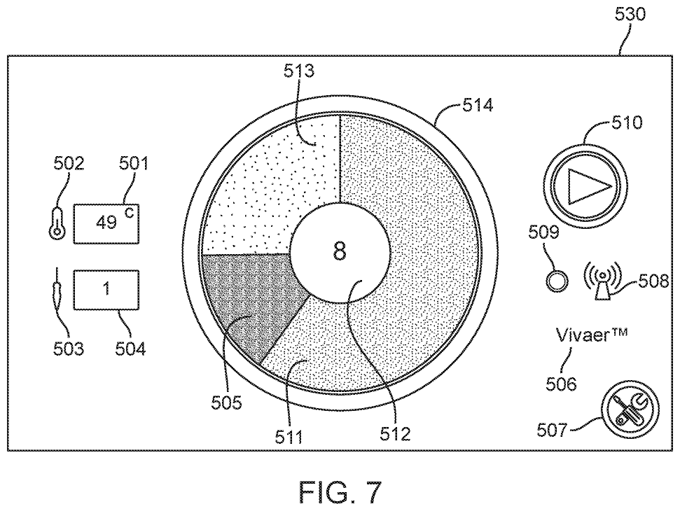

Referring now to FIG. 7, a third screenshot 530 of the default setting screen as displayed on the touchscreen display 108 is illustrated. At a given amount of time into the procedure, the console 102 stops delivering RF energy to the stylus 104, and a cool down phase begins. The cool down phase is shown on the ring 513 as a differently colored or shaded segment, as compared to the RF ON segment 511, which may be called a cool down timer indicator 505. The cool down timer indicator 505 moves around the ring in a clockwise direction until the procedure is complete. Any amount of time remaining in the total procedure, in this case the cool down phase, is indicated by the total treatment time remaining portion of the ring 513.

In the screenshot of the main screen image 530 shown in FIG. 7, the current stage of treatment is illustrated as follows: The central circle total treatment timer 512 shows there are eight seconds remaining in the treatment. The RF ON indicator segment 511 and the cool down timer segment 505 show that the RF delivery portion of the treatment is complete, because the cool down timer segment 505 has started. Since the total treatment time remaining portion of the ring 513 is still visible, this shows the user that the cool down phase is still in process. In a real life scenario, the cool down timer portion 505 would also be moving clockwise, thus easily telling the user what phase the treatment was in. In this case, the total treatment time portion of the ring 513 shows that approximately one fourth of the entire treatment time still remains. The temperature of 49 degrees Celsius in the temperature window also tells the user that the console 102 is in the cooling phase, since the temperature of the stylus 104 has decreased from 60 degrees. Finally, the RF ON indicator light 509 is not illuminated. In summary, the outer ring 513 in FIG. 7 includes three, differently colored sections at this stage: (1) an RF ON treatment stage section 511, indicating that the 18-second RF delivery stage is complete; (2) an RF-off cool down stage section displayed as the cool down timer indicator 505, showing that a portion of the cool down stage is complete; and (3) a remaining treatment portion of the outer ring 513, indicating that a portion of the cool down phase remains to be completed.

In various embodiments, any colors, shades, shapes, graphics and/or the like may be used for the various segments 511, 505 of the outer ring 513. In one embodiment, for example, the RF ON timer indicator 511 is navy blue, the cool down timer indicator 505 is gray, and the total treatment time remaining portion 513 is light blue. Any other colors may be used, however, in alternative embodiments. In another alternative embodiment, the entire ring 513 may be one color, and a line that acts as a timer may move clockwise around the ring 513, similar to a long hand on a clock. In a variation on such an embodiment, the color of the ring 513 behind the moving line may change. Thus, the ring 513 and the segments 511, 505 may have any suitable size, color scheme or configuration.

Additionally, the default (or custom) settings of the console 102 may have any suitable ranges and combinations for the various parameters of the console 102. For example, one timing default setting may have a total treatment time of 30 seconds, an RF ON time of 18 seconds, and a cooling time of 12 seconds. This is but one example, however, and any number of other time settings may alternatively be used. Total treatment time may be in minutes, for example, and each segment of the treatment may be measured in seconds and/or minutes. A default temperature may also be set for RF delivery, for example 60 degrees Celsius as the maximum temperature. Again, any suitable default settings may be set in various embodiments.

Referring now to FIG. 8, as just described, the physician or other user can choose to access a custom treatment screen 630 by touching the custom treatment button 507 on the initial default screen 500 (FIG. 5). In the embodiment shown, the custom treatment screen 630 includes a graphical treatment progress display 600, which is smaller but otherwise the same as the graphical treatment progress display 514 of the default screen 500. The custom treatment screen 630 also includes a timer icon 601, an impedance display 602, a stylus icon 603, a number of treatments indicator 604, a cooling icon 605, a set cooling time window 606, a set RF ON time window 607, a set temperature window 608, an actual temperature indicator 609, a down button 610, a back button 611, a stylus type indicator 612, an RF icon 613, an RF ON indicator 614, a start/stop button 615, an up button 616, an actual power delivery indicator 617, a set power window 618, an RF power icon 619 and a temperature icon 620. As with the previously described default screen 500, the icons and/or indicators of the custom treatment screen 630 may be moved, changed and/or eliminated, according to various alternative embodiments.

Through the custom treatment screen 630, the user can adjust the power (power window 618), temperature (temperature window 608), treatment time (RF on time window 607) and/or cool down time (cooling time window 606), by touching any one of the set windows and then touching the up button 616 and/or the down button 610 to adjust a given value. To set power, for example, the user may touch the power window 618 and then adjust the temperature by pressing the up button 616 or the down button 610. The console 102 may be configured to only allow adjustments within ranges. For example, the power on the console 102 may be selected at 3 W, 4 W or 5 W in one embodiment. Maximum stylus temperature may be selected in a range of 50 degrees Celsius to 70 degrees Celsius in one embodiment. RF energy delivery time (RF ON time) may be selected for between 6 seconds and 18 seconds, in 2-second increments, and cooling time may be selected for between 0 seconds and 12, in 3-second increments, in one embodiment. Any other suitable ranges and combinations of ranges may be used, in alternative embodiments, and those provided here are merely examples.

For the information of the user, the impedance display 602 and stylus usage count 604 are also displayed. The back button 611 can be touched to return to the default screen 500 (FIG. 5). During treatment, the actual RF power 617 and temperature reading 609 are also shown. In alternative embodiments of the custom treatment display screen 630, one or more icons, indicators, buttons and/or windows may be moved, changed or eliminated. In some embodiments, the console 102 might not include any custom settings or custom setting displays. Such a console 102 may be configured to only provide standard treatment parameters.

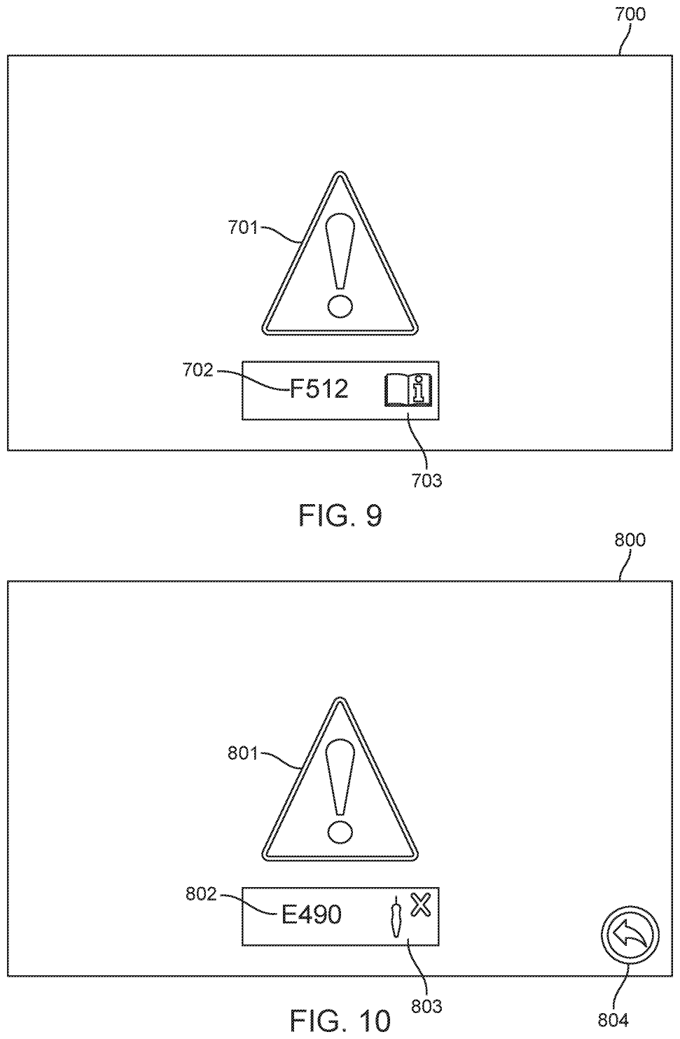

Referring now to FIG. 9, in some embodiments, the console 102 may be programmed to display a fault screen 700 when a serious or fatal error of the system 100 occurs. The fault screen 700 may include, for example, a fault error symbol 701 that indicates a serious error has occurred, rendering the console 102 unusable. The fault screen may also include an error code 702, indicating what kind of error has occurred, and a “refer to IFU” (instructions for use) symbol 703. The fault error symbol 701 may be any color or combination of colors, such as a red triangle with a black exclamation point. In some embodiments, after the fault screen 700 appears, the console 102 may only be used after the user turns the console 102 off and turns it back on again.

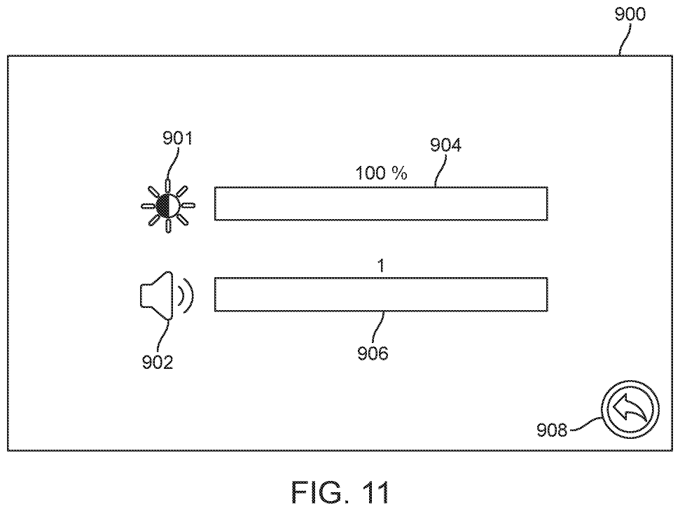

Referring now to FIG. 10, in some embodiments, the console 102 may be programmed to display an error screen 800 when a non-fatal error of the system 100 occurs. The error screen 800 may include, for example, a caution symbol 801 that indicates an error has occurred. The error screen may also include an error code 802, indicating what kind of error has occurred, an error symbol 803, and/or a back button 804. In the example of FIG. 10, the error symbol 803 indicates a problem with the stylus 104 that is inserted in the console 102, as might occur for example if a used or incompatible stylus is inserted. The caution symbol 801 may be any color or combination of colors, such as a yellow triangle with a black exclamation point. In this embodiment, the user may touch the back button 804 to return to the previous screen being used when the error occurred, and the user may fix the error at that screen.

FIG. 11 is a screenshot of a settings screen 900, which may be provided on the display 108 of the console 102. In this embodiment, the settings screen 900 includes an adjust brightness icon 901, an adjust volume icon 902, a sliding bar brightness control 904 and a sliding bar volume control 906. The brightness control 904 and the volume control 906 may have any color or combination of colors. In some embodiments, a numerical indication of brightness and volume may also be included. The settings screen 900 also includes a back button 904 to allow the user to return to a previous screen. The settings screen 900 may be available in some embodiments, even if the user is not permitted to select custom treatment settings.

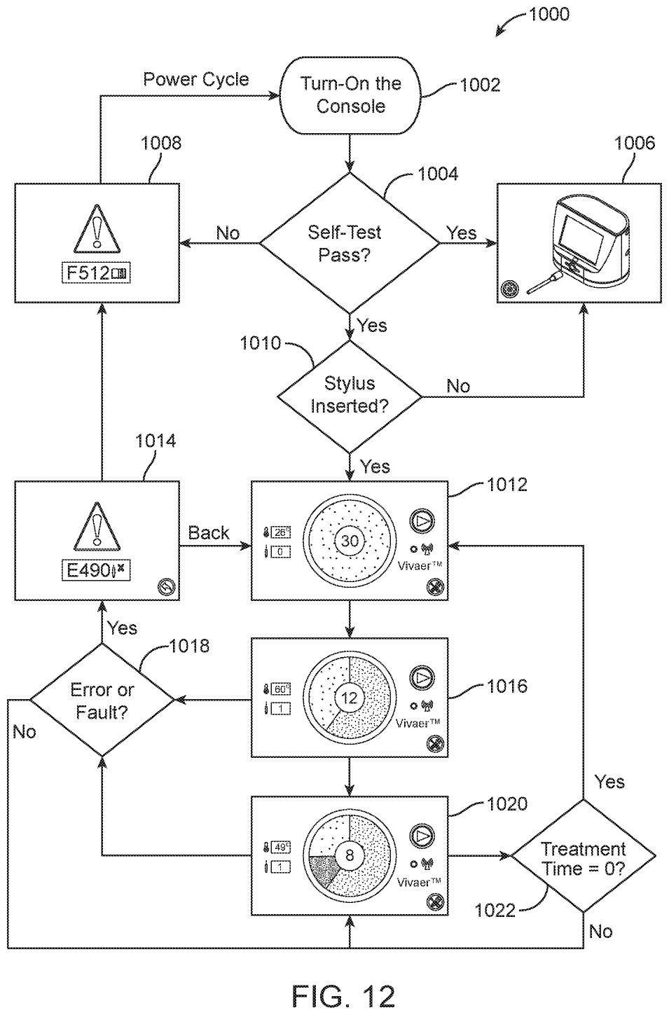

Referring now to FIG. 12, a method 1000 of using the console 102 of the electrosurgery system 100 is illustrated. First, the user turns on 1002 the console 102. The console 102 performs a self-test 1004. If the self-test passes, the console 102 displays the standby screen 1006. If the console 102 fails the self-test, the fault screen is displayed 1008. In some embodiments, the fault screen can only be cleared by power cycling the console 102 to repeat the self-test routine. Next, assuming the self-test is passed, the user plugs the stylus 104 into the console 102. If the correct stylus is inserted 1010, the console 102 shows the default main screen 1012. The user presses the start/stop button or a foot switch, and the RF energy delivery portion of the treatment starts 1016, followed by the cooling portion 1020. When the treatment is completed 1022, the screen returns to the default main screen 1012. If an error occurs during treatment 1018, the error screen is shown 1014. The user can press the back button on the error screen to return to the default main screen 1012. If the error is fatal, the screen changes to the fault screen 1008. At various points the in method 1000, the user may also select the custom treatment screen (FIG. 8) by pressing the custom treatment button 507 on the default screen. In alternative embodiments, various screens and/or method steps may be changed or eliminated.

Although this application is believed to be complete and accurate, any suitable changes may be made to any of the described embodiments and features described above, without departing from the scope of the invention.

We claim:

1. A console for an electrosurgical device, the console comprising:
a housing;
an energy generator in the housing;
a computer processor in the housing;
a connector on the housing, for connecting the console with an energy delivery treatment device;
a touchscreen display on the housing; and
a non-transitory computer readable medium in the computer processor, containing computer-executable programming instructions for providing an active display on the touchscreen display, the active display comprising:
a central circle;
a countdown timer in the central circle for counting down a total time remaining in an energy delivery procedure; and
an outer ring around the central circle, configured to indicate the total time remaining in the energy delivery procedure, a remaining time in an energy delivery stage of the energy delivery procedure, and a remaining time in a cooling stage of the energy delivery procedure.

2. The console of claim 1, wherein the energy generator comprises a radiofrequency (RF) generator configured to deliver at least one of monopolar or bipolar RF energy to the energy delivery treatment device.

3. The console of claim 1, wherein the outer ring is configured to change colors in a clockwise direction to indicate elapsed time of the energy delivery procedure.

4. The console of claim 1, wherein the active display further comprises:
a start/stop button; and
a custom treatment button, wherein the start/stop button and the custom treatment button are configured to be activated by touch.

5. The console of claim 1, wherein the active display further comprises at least one of a temperature display, an energy on indicator, or a procedure count indicator, indicating a number of procedures completed using the energy delivery treatment device.

6. The console of claim 1, wherein the active display further comprises a custom treatment button that, when activated, causes the display screen to display a custom treatment screen.

7. The console of claim 6, wherein the custom treatment screen comprises at least one of a set cooling time window, a set power window, a set temperature window or a set procedure time window.

8. The console of claim 1, wherein the connector on the housing is configured to accept only one predefined type of energy delivery treatment device.

9. The console of claim 1, wherein the non-transitory computer readable medium in the computer processor further comprises an energy delivery treatment device identifier for identifying the energy delivery treatment device that is connected to the console.

10. A method for performing an energy delivery therapy on nasal airway tissue in a patient, the method comprising:
activating a console attached to an energy delivery stylus;
advancing a distal end of the energy delivery stylus into a nostril of the patient;
delivering energy from the energy delivery stylus to treat the nasal airway tissue;
watching a display on a display screen of the console, wherein the display comprises:
a central circle;
a countdown timer in the central circle for counting down a total time remaining in the energy delivery therapy; and
a moving outer ring around the central circle, configured to indicate the total time remaining in the energy delivery therapy, a remaining time in an energy delivery stage of the energy delivery therapy, and a remaining time in a cooling stage of the energy delivery therapy; and
removing the energy delivery stylus from the nostril after completion of the cooling stage.

11. The method of claim 10, wherein delivering energy comprises delivering radiofrequency energy, and wherein the energy delivery stylus comprises a radiofrequency stylus.

12. The method of claim 10, wherein delivering energy comprises starting the delivery of energy by at least one of touching a start/stop button on the display screen or depressing a foot pedal coupled with the console.

13. The method of claim 10, further comprising observing at least one of a displayed temperature, a displayed number of procedures performed with the energy delivery stylus, an energy on indicator, or a stylus identifier on the display screen.

14. The method of claim 10, further comprising:
touching a custom treatment button on the display screen to switch to a custom treatment display; and
setting at least one of a temperature, a power, a therapy time, or a cooling time, by touching at least one control button on the custom treatment display.

15. The method of claim 10, further comprising:
viewing an error display on the display screen; and
touching a back button on the display screen to return to a previous screen.

16. The method of claim 10, further comprising:
viewing a settings display on the display screen; and
adjusting at least one of brightness or volume of the console by touching at least one controller on the display screen.

17. The method of claim 10, further comprising, before removing the energy delivery stylus from the nostril:
moving the distal end of the energy delivery stylus to a different location in the nasal airway;
delivering energy from the energy delivery stylus to treat nasal airway tissue at the different location; and
watching the display on the display screen of the console.

18. The method of claim 17, further comprising repeating the moving, delivering and watching steps as often as desired to perform the energy delivery therapy on the nasal airway tissue.

19. The method of claim 10, further comprising:
attaching the energy delivery stylus to the console; and
waiting for the console to confirm that the energy delivery stylus is approved for use, wherein the console activates after it confirms the energy delivery stylus is approved.
